(19) United States
(12) Patent Application Publication
(10) Pub. No.: US 2024/0350019 A1
(43) Pub. Date: Oct. 24, 2024

(54) WEARABLE PHYSIOLOGICAL MONITORING SYSTEM

(71) Applicant: TOPSIGHT MONITORING LTD, Tel Aviv (IL)

(72) Inventors: Revital PERY-SHECHTER, Rishon Lezion (IL); Michal BALBERG, Jerusalem (IL)

(73) Assignee: TOPSIGHT MONITORING LTD, Tel Aviv (IL)

(21) Appl. No.: 18/687,039

(22) PCT Filed: Aug. 30, 2022
(86) PCT No.: PCT/IL2022/050950

§ 371 Date: Feb. 27, 2024

Related U.S. Application Data
(60) Provisional application No. 63/238,226, filed on Aug. 30, 2021.

(51) Int. Cl. A61B 5/0205; A61B 5/00; A61B 5/02; A61B 5/021; A61B 5/026; A61B 5/1455

(52) U.S. Cl. CPC A61B 5/0205; A61B 5/02007; A61B 5/0261; A61B 5/14551; A61B 5/746; A61B 5/02125

(57) ABSTRACT

A physiological parameters monitoring device and methods are disclosed utilizing at least one coherent light source configured to emit light in one or more wavelengths onto a tissue of an examined subject at a measurement point. at least one light detector configured to measure intensity of light received from the tissue, and generate measurement data/signals indicative thereof. and a control unit configured to process the measurement data/signals generated by the at least one light detector and simultaneously determine therefrom a pulsating blood flow signal and a pulsating blood volume signal, and determine from the pulsating blood flow and volume signals a blood pressure measure of the examined subject at the measurement point as a function of time.

### TECHNOLOGICAL FIELD

The present invention is generally in the field of medical measuring devices, and particularly relates to wearable medical devices usable for measuring physiologic signals.

### BACKGROUND ART

This section intends to provide background information concerning the present application, which is not necessarily prior art.

Vital signs monitoring (VSM) devices are utilized for monitoring physiologic vital signs in medical and/or home care settings. Such VSM devices are typically configured to measure basic body functions, such as body temperature, pulse rate, respiration rate, and blood pressure. The early detection/monitoring of evolving medical problems by such VSM devices in medical and/or home settings is very important for carly diagnosis of various medical conditions.

The vital signs monitoring (VSM) devices market is growing rapidly inter alia due to the increase in cardiovascular disorders (e.g., hypertension, high blood pressure (BP) risks), the increase in geriatric population, in trauma conditions and other health conditions such as COPD, sleep apnea, infectious diseases, obesity and diabetes.

Cardiovascular diseases are the leading cause of death (accounting for about 32% of all deaths) globally, having a profound impact on peoples' wellbeing and lifespan. The performance of the cardiovascular system relies on biomechanical interactions between the heart, the vascular networks, and the microvascular beds (e.g., cardiac contraction, ventriculo-vascular coupling, artery stiffness, and microvasculature properties). Various factors can contribute to the incidence and progression of cardiovascular disease, resulting in failure or ineffective delivery of oxygenated blood to organs and tissues. Studies show that analysis of blood pulse waves can be used to provide valuable prognostic information of major risk factors.

In each heart beat the heart ejects a bolus of blood into the arteries, inducing a change in the local flow of blood within the vascular compartments. This induced change propagates through the vasculature at a speed determined by, among other parameters, the resistivity of the vascular network. As a result of the movement of blood cells, induced by the bolus of blood, a pressure gradient is induced inside the blood vessels. This pressure gradient first induces dilation and then constriction of the vessels. As the blood vessels branch into smaller and smaller compartments, the resistivity of the network is increased. As a result of resistivity (or vascular impedance) mismatches, the ejected blood is partially transmitted and partially reflected at branching points of the blood vessels, causing reflection of blood cells, which propagate in an opposite direction to the heart induced flow. The reflected blood propagates backwardly in the blood vessels, causing an increase in the pressure and a decreased in flow. As pulsatile blood propagates from the central arteries to the periphery vessels, the increased resistance in peripheral vessels induces more reflection waves.

Blood flow parameters can be measured using several techniques known in the art, such as for example, laser doppler (LD), ultrasound modulated light (UTL), diffuse correlation spectroscopy (DCS) or speckle based flowmetry, to name a few. Blood pressure can be estimated non-invasively using photoplethysmography (PPG) a combination of Electrocardiogramand PPG, or other techniques that are known in the art, including invasive measures (sec e.g. Wang, G., Atef, M., & Lian. Y. (2018) “Towards a continuous non-invasive cuffless blood pressure monitoring system using PPG: Systems and circuits review”, IEEE Circuits and systems magazine. 18 (3). 6-26).

PPG is a non-invasive optical technique usable for measuring subtle variations in blood volume in the examined tissue via light absorption therein. The PPG signal comprises a slowly varying (DC) signal associated with non-pulsatile blood, and a pulsatile (AC) waveform that rises and falls during the systolic phase and then slightly increases again during the diastolic phase of each heartbeat, and which is related to the increase/decrease of blood volume within the examined vasculature.

Few solutions known from the patent and academic literature, the disclosures of which are incorporated herein by reference, are briefly described hereinbelow.

US Patent Publication No. 2013/204112 discloses methods and apparatus for measuring perfusion using transmission laser speckle imaging. The apparatus comprises a coherent light source and a detector configured to measure transmitted light associated with an unfocused image at one or more locations. The coherent light source and detector are positioned in a transmission geometry. The apparatus further comprises means for securing the coherent light source and the detector to the tissue sample in a fixed transmission geometry relative to the tissue sample. The apparatus may further comprise at least one processor to receive information from the detector and process detected variations in transmitted light intensity to determine a single metric of perfusion. The method may comprise the steps transilluminating a tissue sample with coherent light, recording spatial and/or temporal variations in the transmitted light signal, determining speckle contrast value(s), and computing a metric of perfusion.

A work by M. Ghijsen et al (“Wearable speckle plethysmography (SPG) for characterizing microvascular flow and resistance”, Vol. 9, No. 8, 1 Aug. 2018, BIOMEDICAL OPTICS EXPRESS 3937), introduced a modified form of laser speckle imaging (LSI) referred to as affixed transmission speckle analysis (ATSA) that uses a single coherent light source to probe two physiological signals: one related to pulsatile vascular expansion (classically known as the photoplethysmographic (PPG) waveform) and one related to pulsatile vascular blood flow (named here the speckle plethysmographic (SPG) waveform). The PPG signal is determined by recording intensity fluctuations, and the SPG signal is determined via the LSI dynamic light scattering technique. These two co-registered signals are obtained by transilluminating a single digit (e.g. finger) which produces quasi-periodic waveforms derived from the cardiac cycle. Because PPG and SPG waveforms probe vascular expansion and flow, respectively, in cm-thick tissue, these complementary phenomena are offset in time and have rich dynamic features.

US Patent Publication No. 2021/022623 discloses systems and methods for determining physiological information in a subject. The system includes; a light source positioned along a first location outside of the subject; a photo-sensitive detector positioned along a second location outside of the subject and configured to detect scattered light and generate a signal; a processor having a program and a memory, wherein the processor is operably coupled to the detector and configured to receive and store the signals generated over a period of time; wherein the processor is programmed to derive contrast metrics from the stored signals, calculate a waveform from the contrast metrics, decompose the waveform into basis functions and respective amplitudes, and compare the basis function amplitudes to determine the physiological information.

A device that can measure micro-vascular blood flow parameters simultaneously (whether pulsatile or non-pulsatile) with blood pressure parameters enables measurement of vascular reactivity to changes in blood pressure. Such a device also enables measurement of the response of the vascular system to various physiologic changes, in particular during shock or sepsis. Simultaneous measurements of both blood flow and pressure parameters from the vasculature of the brain, can provide a measure of the state of cerebral autoregulation, which is a measure indicative of brain health. Currently, cerebral autoregulation or cerebro-vascular reactivity is non-invasively measured indirectly, with one sensor estimating continuous blood pressure modulation (either invasively or non-invasively) and another sensor measuring a surrogate parameter of blood flow non-invasively (see e.g., U.S. Pat. No. 8,556,811). A single sensor can also be used to measure blood pressure modulation and blood flow

### General Description

There is a great need to non-invasively, accurately and reliably, measure physiological parameters, such as vital clinical signs in a simple, lightweight, easy to use and interpret way. In particular, in many medical monitoring scenarios it is important to measure blood flow and blood pressure parameters in a simple to use, continuous and non-invasive manner. For example, optical measurement devices can be miniaturized to provide lightweight and compact measurement units that can be comfortably worn on the body of an examined subject for continuous measurement of both blood flow and blood pressure parameters.

The present application discloses monitoring embodiments (e.g., wearable), that measure optical signals propagating through a body part of an examined subject. The physiological parameters monitoring techniques and implementations that are disclosed herein enable to measure in some embodiments, both, blood flow, blood volume and blood pressure, parameters, simultaneously or independently, within the same body part, or in different body parts, of the user/examined subject. In a broad aspect, embodiments disclosed herein provide measurement arrangements configured to acquire optical measurement data/signal obtained from an examined tissue and extract therefrom at least one of blood volume related pulse wave signals (also referred to herein as pulsating blood volume signal) and blood flow related pulse wave signals (also referred to herein as pulsating blood flow signal), and use the same to determine a local pulse wave velocity (PWV) measure. The determined PWV measure can be then used to determine a blood pressure measure for the examined subject.

In some embodiments a correlation index between either the blood flow or blood volume with blood pressure parameters is calculated over predetermined periods of time. The correlation index can be displayed or communicated to the user, and/or to a caregiver, either graphically, numerically or audibly e.g., using sounds. The physiological parameters monitoring techniques/implementations hereof can be thus configured to issue alerts indicative in real-time of evolving medical conditions/problems e.g., utilizing one or more correlation indices to identifying evolving states of shock, trauma, and/or inadequate tissue perfusion, as they occur.

An optical assembly is accordingly used in some embodiments to measure blood flow and/or volume related parameter(s), and/or changes in said blood flow and/or volume related parameters, within the microvasculature of the body part of the examined subject. Optionally, but in some embodiments preferably, the optical assembly is based on laser Doppler technology, but can also incorporate other methods for blood flow measurements. The same optical assembly is used in some embodiments to measure blood volume, blood pressure, and/or changes in blood pressure e.g., based on acquired PPG signals.

Alternatively, or additionally, an auxiliary assembly is used to measure the blood pressure, and/or to provide information from which the assembly can extract a measure of the blood pressure of the examined subject. In another possible embodiment, an ultrasound modulated light system is employed to extract a measure of microvascular blood flow within a tissue of the examined subject.

In other possible embodiments blood flow measure of the examined subject is determined by monitoring changes over time of spectral components (e.g., using short-time fast Fourier transform-sFFT analysis) of the measured optical signals (e.g., intensity of received light on a photodiode). Accordingly, optical signals measured by light transmittance or reflectance measurement setup are used in some embodiments to extract both blood volume pulse wave signals (also referred to herein as blood volume related signals (e.g., PPG) of the examined subject, and blood flow pulse wave signals (also referred to herein as blood flow related signals). Optionally, spectral analysis of the optical signal, or by other methods known in the art as described below. signals, or alternatively, on predefined consecutive time windows, where each predefined time window of the measured optical signals may comprise a plurality of the blood pulse waves identified in the measured optical signals.

This way, both changes in blood flow and blood volume can be determined simultaneously, and a measure corresponding to the relationship (e.g., a ratio and/or a delay between them) between the blood volume and blood flow related pulse wave signals can be extracted. Such a measure can be defined as the delay time between the (e.g., locally maximal) peak of the blood flow pulse wave related signal and the (e.g., locally maximal) peak of the blood volume pulse wave related signal, within each identified pulse in the signal wave, or their relative slopes (e.g., as measured before, upto one sample point, or after, at least one sample point, the first/primary and/or secondary peak in each identified one or more blood volume/flow related pulse wave signals) or primary/secondary peak amplitudes of the two pulse wave signals, or the first or second derivatives of the blood volume and blood flow related pulse wave signals, or a measure of the correlation between the two signals, or of the coherence between the two signals. In possible embodiments a measure corresponding to the relationship (e.g., ratio) between the blood volume and blood flow related signals is determined from derivatives, or from the relative derivatives, between these signals, or from both their derivatives and their relative derivatives.

The measure corresponding to the relationship between the blood volume and blood flow related signals can be used to alert that undesired medical conditions are evolving which requires caregivers/practitioners intervention

Accordingly, embodiments disclosed herein can be implemented utilizing a single coherent light source and a single light detector. One aspect of the subject matter disclosed herein relates to a physiological parameters monitoring (e.g., wearable) device comprising at least one coherent light source configured to emit light in one or more wavelengths onto a tissue of an examined subject at a measurement point, at least one light detector configured to measure intensity of light received from the tissue and generate measurement data/signals indicative thereof, and a processor and/or control unit configured to process the measurement data/signals generated by the at least one light detector and simultaneously determine therefrom a pulsating blood flow signal and a pulsating blood volume signal, and determine from the pulsating blood flow and volume signals a blood pressure measure of the examined subject at the measurement point as a function of time. Optionally, the light emitted by the light source in at least one of the one or more wavelengths is a coherent light. The processor/control unit is configured in some embodiments to acquire the measurement data/signals at a sample rate greater than 100 Hz.

In possible embodiments the blood pressure measure is determined from the time derivative of the pulsating blood volume signal and from an amplitude of the pulsating blood flow signal measured at two separate time points. Optionally, but in some embodiments preferably, the two separate time points are separated by at least two sample points.

The processor/control unit is configured in some embodiments to determine a pulse-wave-velocity (PWV) time function measure M(t.t+Δt), based on the determined pulsating blood flow and volume signals, and use the PWV time function measure to compute the blood pressure measure of the examined subject. The processor/control unit can be configured to receive blood pressure measurement data/signals generated by an auxiliary sensor device associated with a different tissue of the examined subject, and use the received blood pressure measurement data/signals to determine a calibration biasing value β and a calibration factoring value ϵ for the computation of the blood pressure measure of the examined subject from the determined PWV time function measure. For example, the processor/control unit can be configured to determine the blood pressure measure of the examined subject by computation of the expression ϵ*M(t.t+Δt)²+β.

The device comprises in some embodiments an ultrasound transducer configured to induce acoustic signals in the examined tissue for modulating the light received by the at least one light detector.

The processor/control unit is configured in possible embodiments to determine correlation between the determined blood flow or volume signal and the determined blood pressure measure, and determine based thereon a measure of vascular reactivity or autoregulation of the examined tissue. Optionally, but in some embodiments preferably, the processor/control unit is configured to issue alerts indicative of changes in perfusion state of a body part of the examined subject based on the determine correlation. For example, the processor/control unit can be configured to issue the alerts for indicating of at least one of the following states: onset of severe sepsis, shock, trauma, and/or inadequate perfusion.

The processor/control unit is configured in some embodiments to determine a vascular reactivity measure based on the determined blood pressure measure and the determined blood flow signal.

In possible embodiments the processor/control unit is configured to issue alerts indicative of changes in a clinical or physiological state of the examined tissue based on one or more of the pulsating blood volume and/or flow signals, and/or the determined blood pressure measure, and/or a correlation thereof. The processor/control unit can be configured to issue the alerts based on comparison of the determined pulsating blood flow and/or volume signals and/or the determined blood pressure measure, or their correlation, or a combination thereof. to corresponding measurement data/signals retrieved from a database of such measurements. Additionally or alternatively, the processor/control unit is configured to issue the alerts based on machine-learning and/or deep-learning process utilizing the measurement data/signals recorded in the database of measurements.

The device comprises in some embodiments a blood flow controlling element configured to locally affect perfusion in the examined tissue responsive to instructions from the control unit. The processor/control unit can be configured to operate the blood flow controlling element based on the determined pulsating blood flow and/or volume signals and/or the determined blood pressure measure. Optionally, the blood flow controlling element is configured to controllably apply pressure to the examined tissue, and/or cool or heat the examined tissue.

The device comprises in some embodiments a temperature sensor configured to measure body temperature of the examined subject.

In possible embodiments the coherent light source is configured to selectively emit the light in two or more different wavelengths, and the control unit is configured to determine oxygen saturation of the examined subject based on the blood pulse wave determined for the two or more different wavelengths.

In another aspect the subject matter disclosed relates to a method of measuring physiological parameters of an examined subject. This comprises illuminating a tissue by at least one coherent light source, measure intensity of light received from the tissue and generating measurement data/signals indicative thereof, processing the measurement data/signals and simultaneously determining therefrom a pulsating blood flow signal and a pulsating blood volume signal, and determining from the pulsating blood flow and volume signals a blood pressure measure of the examined subject at the measurement point as a function of time. The method optionally comprises determining the blood pressure measure from a time derivative of the pulsating blood volume signal and from an amplitude of the pulsating blood flow signal measured at two separate time points. The method optionally comprises selecting the two separate time points to obtain a separation of at least two sample points therebetween.

In some embodiments the method comprises determining a pulse-wave-velocity (PWV) time function measure M(t.t+Δt). based on the determined pulsating blood flow and volume signals, and computing the blood pressure measure of the examined subject based on the PWV time function. The method can comprise receiving externally produced blood pressure measurement data/signals associated with a different tissue of the examined subject, and determining based on the externally produced blood pressure measurement data/signals a calibration biasing value β and a calibration factoring value ϵ for the computation of the blood pressure measure of the examined subject from the determined PWV time function measure. The method comprises in some embodiments determining the blood pressure measure of the examined subject from the expression ϵ*M(t.t+Δt)²+β.

The method comprises in possible embodiments determining correlation between the determined blood flow or volume signal and the determined blood pressure measure, and determining based on the correlation a measure of vascular reactivity or autoregulation of the examined subject. The method can further comprise issuing alerts indicative of changes in perfusion state of a body part of the examined subject based on the determine correlation. The issuing of the alerts is used in some embodiments for indicating at least one of the following states: onset of severe sepsis, shock, trauma, and/or inadequate perfusion.

The method comprises in possible embodiments determining a vascular reactivity measure based on the determined blood pressure measure and the determined blood pulsating blood flow signal.

The method may include issuing alerts indicative of changes in a clinical or physiological state of the examined subject based on one or more of the pulsating blood volume and/or flow signals, and/or the determined blood pressure measure, and/or a correlation thereof. The method comprises in some embodiments issuing the alerts based on comparison of the determined pulsating blood flow and/or volume signals and/or the determined blood pressure measure, or their correlation, or a combination thereof, to corresponding measurement data/signals retrieved from a database of such measurements. Additionally or alternatively, the method comprises issuing the alerts based on machine-learning and/or deep-learning process utilizing the measurement data/signals recorded in the database of measurements.

The method can include locally affecting perfusion in the examined tissue based at least partially on the determined pulsating blood flow and/or volume signals and/or the determined blood pressure measure. The method may comprise measuring body temperature of the examined subject.

In some possible embodiments the method comprises selectively emitting the light in two or more different wavelengths, and determining oxygen saturation of the examined subject based on the determined pulsating blood flow and/or volume signals determined for the two or more different wavelengths.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings. Features shown in the drawings are meant to be illustrative of only some embodiments hereof, unless otherwise implicitly indicated. In the drawings like reference numerals are used to indicate corresponding parts, and in which:

FIG. 1 is a block diagram schematically illustrating an optical measurement setup according to some possible embodiments;

FIGS. 2A and 2B are block diagrams schematically illustrating designs of optical measurement physiological parameters monitoring devices according to some possible embodiments, wherein FIG. 2A demonstrates use of transmission geometry and FIG. 2B demonstrates use of reflection geometry;

FIGS. 3A and 3B are block diagrams schematically illustrating physiological parameters monitoring devices utilizing ultrasound modulated light measurements according to some possible embodiments, wherein FIG. 3A demonstrates use of a transmission geometry for the ultrasound modulated light measurements and FIG. 3B demonstrates use of a reflection geometry for the ultrasound modulated light measurements;

FIGS. 4A and 4B schematically illustrate physiological parameters monitoring device implementations according to some possible embodiments, wherein FIG. 4A is a functional block diagram and FIG. 4B demonstrates the physiological parameters monitoring device positioned on the car auricle (pinna) of the examined subject;

FIG. 5 shows exemplary blood flow related pulse wave signal (e.g., PBFv signal) and blood volume related pulse wave signal (e.g., PPG signal), and various parameters extracted therefrom in physiological parameters monitoring device implementations according to possible embodiments;

FIG. 6 shows an exemplary blood volume pulse wave related signal (e.g., PPG signal) and parameters extracted therefrom in physiological parameters monitoring device implementations according to possible embodiments; and

FIGS. 7A and 7B are flowcharts schematically illustrating blood pressure measurement procedures according to some possible embodiments.

### DETAILED DESCRIPTION OF EMBODIMENTS

One or more specific and/or alternative embodiments of the present disclosure will be described below with reference to the drawings, which are to be considered in all aspects as illustrative only and not restrictive in any manner. It shall be apparent to one skilled in the art that these embodiments may be practiced without such specific details. In an effort to provide a concise description of these embodiments, not all features or details of an actual implementation are described at length in the specification. Elements illustrated in the drawings are not necessarily to scale, or in correct proportional relationships, which are not critical. Emphasis instead being placed upon clearly illustrating the principles of the invention such that persons skilled in the art will be able to make and use such physiological parameters monitoring implementations, once they understand the principles of the subject matter disclosed herein. This invention may be provided in other specific forms and embodiments without departing from the essential characteristics described herein.

Disclosed herein is a compact sized wearable physiological parameters monitoring device, method and system, for measuring various blood characteristics/parameters, e.g., blood pressure, blood flow, and/or possibly for obtaining other parameters such as blood vessel flexibility and resistivity based on the blood characteristics/parameters. One or more light sources, at least one of which is coherent, are used to illuminate a selected body region/tissue. The light source can be operated continuously, intermittently or modulated at a specific frequency f0. An optical detector is used to measure intensity of light received from the examined body region/tissue. The light detector can be positioned at the same side of the one or more light sources, or on the opposite side of the examined body region/tissue, such that the optical detector collects the light reflected from, or transmitted through, the examined body region/tissue for continuously generating measurement data/signals indicative of the intensity of the transmitted/reflected light.

One or more processors and memories can be used to process the measurement data/signals generated by the detector and determine one or more different blood characteristics/parameters therefrom e.g., pulse wave signals related to/indicative of the blood volume pulse waves (e.g., PPG) and/or pulse wave signals related to the blood flow e.g., indicative of the blood flow (velocity), in the examined body region/tissue. The processing of the measurement data/signals may use model-based processing utilizing machine-learning and/or deep-learning techniques to determine the blood characteristics/parameters from the measurement data/signals generated by the detector.

In some embodiments the measurement data/signals is processed to extract therefrom blood volume related pulse wave signals (also referred to herein as a blood volume measure e.g., PPG), and/or the blood flow related pulse wave signals (also referred to herein as blood flow measure e.g., PBFv). For example, in possible embodiments a local frequency domain extremum is detected for each pulse in the optical signal, or for a predefined time windows (e.g., a moving time window used to process the extracted optical signals, with or without overlaps), each comprising one or more optical signals, and the blood flow related pulse wave signals are determined based on changes occurring over time in the detected frequency domain extremums.

The determined/measured blood volume and blood flow related pulse wave signals can be then used to determine various physiological measures/parameters of the examined subject e.g., blood pressure measures, blood volume measures, heart rate measures, pulse pressure measures, respiratory rate measures, oxygen saturation, cardiac output and/or other measures. For example, in possible embodiments time differences between main and/or secondary peaks in the determined blood volume and blood flow related pulse wave signals are used to determine blood pressure measures for the examined subject.

Optionally, but in some embodiments preferably, relations between characteristic features and/or spectral components of the determined blood volume and flow related pulse wave signals are used as indicators for identification and/or alarming about evolvement of alerting medical conditions in the examined subject e.g., shock, trauma, severe sepsis, inadequate perfusion, or vascular reactivity. The relations between the determined blood volume and flow related signals or pulse wave signals can utilize a computed correlation between the signals or pulse wave signals, and/or ratios of their primary and/or secondary peak amplitudes, and/or ratios of their slopes near the primary and/or the secondary peaks (e.g., within the range of the sample that is closest to the peak), and/or ratios of their first (or second) derivatives (or relative derivatives), and/or measures of coherence therebetween.

For an overview of several example features, process stages, and principles of the disclosed subject matter, the monitoring device examples illustrated schematically and diagrammatically in the figures are intended to provide wearable physiological parameters monitoring device implementation. These devices are shown as one example implementation that demonstrates a number of features, processes, and principles used for body/physiologic parameters monitoring, but they are also useful for other applications and can be made in different variations. Therefore, this description will proceed with reference to the shown examples, but with the understanding that the invention recited in the claims below can also be implemented in myriad other ways, once the principles are understood from the descriptions, explanations, and drawings herein. All such variations, as well as any other modifications apparent to one of ordinary skill in the art and useful in physiological parameters monitoring applications may be suitably employed, and are intended to fall within the scope of this disclosure.

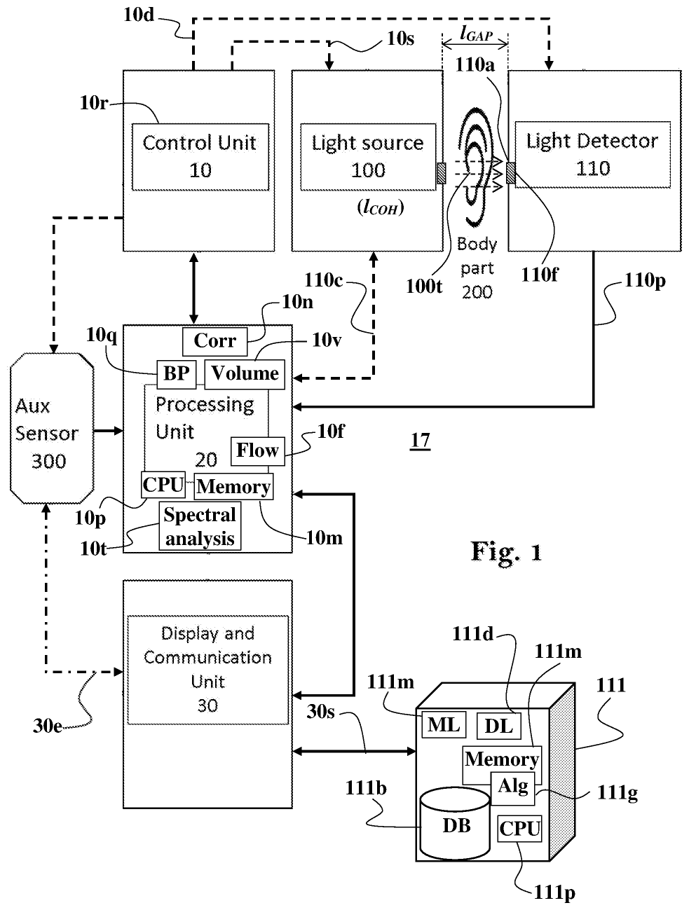

FIG. 1 schematically illustrates an optical measurement setup/system 17 usable for measuring various body parameters of an examined subject (not shown). In possible embodiments the measurement setup/system 17 is configured to determine measures and/or parameters, indicative of one or more of the following: blood flow, blood volume, blood pressure, heart rate, respiratory rate, cardiac output and/or oxygen saturation. The measurement setup/system 17 can however be used to measure other physiological signals, in particular of the micro-vasculature.

The measurement setup/system 17 comprises a control unit 10 configured to control the operation of setup/system 17 and various functions thereof, a light source 100, and a light detector 110. The control unit 10 can be coupled by wires, or wirelessly, to the light source 100 and/or the light detector 110, for provision of respective control data/signals 10s,10d thereto for carrying out various measurements. The control unit 10 comprises in some embodiments an electronic circuit 10r, and/or one or more processors 10p and memories 10m for storing and executing program code/instructions and other data required to operate and control the measurement setup/system 17.

For example, the control unit 10 is configured in some embodiments to control at least one of the following: the measurement(s) repetition rate, the electric supply and/or optical power of optical signals generated by the light source 100, the operating parameters of the light detector 110, such as, but not limited to, gain or filtering. In possible embodiments, the control unit 10 includes a power source (e.g., battery, and/or suchlike power source).

Optionally, but in some embodiments preferably, the light source 100 is operated in the visible to near infrared spectrum (e.g., in the 500 nm to 1000 nm wavelength range). A single wavelength, or several wavelengths, can be emitted by the light source 100, sequentially or simultaneously, using several light emitting sources. It is noted in this respect that though the light source 100 can be configured to emit light in multiple different wavelengths, the light thereby emitted in at least one of said multiple different wavelengths is required to be a coherent light having a suitable a coherence length (l_{COH}). The light source 100 can be implemented utilizing one or more laser devices, and/or laser diodes, and/or light emitting diodes, at least one of which having a coherence length l_{COH} longer/greater than the optical path of light between the light source 100 and the light detector 110 as it passes through the examined tissue 200 (l_{COH}>l_{GAP}, in particular l_{COH}>optical path between 100 and 110). The light source 100 can be operated continuously, intermittently or modulated at a specific frequency f0.

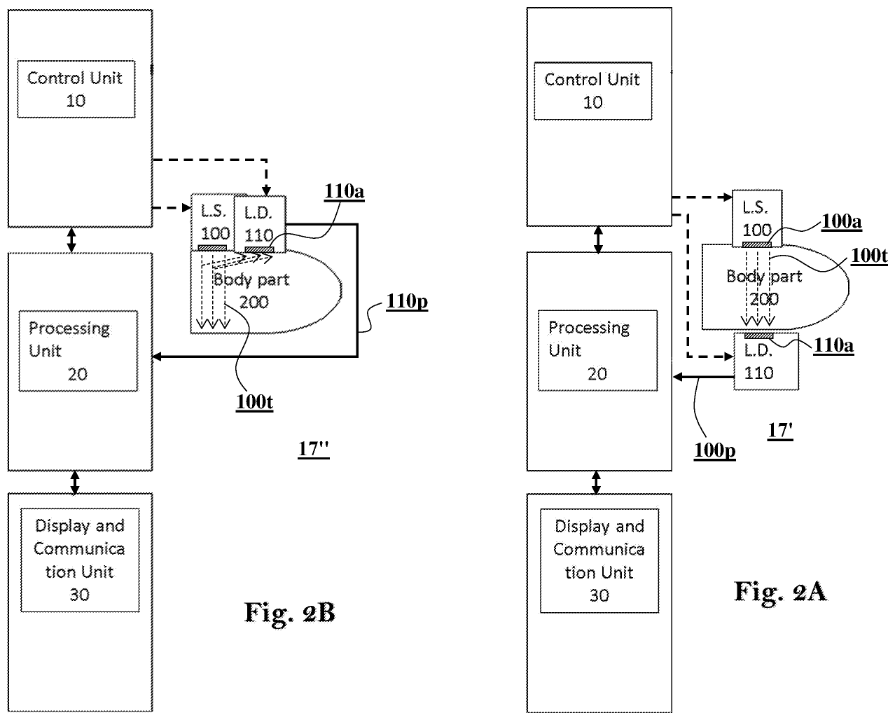

For example, but without limiting, in possible embodiments utilizing transmission geometry (such as exemplified in FIG. 2A), the coherence length l_{COH} of the light 100t produced by the light source 100 in at least one wavelength can be longer than 3 cm, whereas for possible embodiments utilizing reflection geometry (such as exemplified in FIG. 2B), the coherence length l_{COH} of the light 100t produced by the light source 100 in said at least one wavelength can be longer than 10 cm. Optionally, and in some embodiment preferably, the coherence length l_{COH} of the light 100t produced by the light source 100 in said least one wavelength is longer than 30 cm.

The light source 100 can be optically coupled to a tissue/body part 200 (e.g., skin) of the examined subject e.g., using optical fibers (not shown), and/or other optical elements (not shown), such as lenses or prisms, or by free-space coupling. The examined tissue/body part 200 can be for example, an earlobe, an ear auricle, a finger or a toe, a wrist, a nostril, the forchead, limbs, chest or abdomen. The light detector 110 is implemented in possible embodiments by a photodiode, and/or an avalanche photodiode, and/or a photomultiplier, and/or a camera, and/or by an array of such (or different) light detectors. A detector array with optical filters (not shown) can be used in possible embodiments wherein the protocol for illuminating the examined tissue/body part 200 includes simultaneous use of multiple light sources.

The aperture 110a of the light detector 110 can be configured/positioned to collect ballistic photons (photons that do not undergo scattering), or near ballistic photons, or it may be configured/positioned to collect scattered photons, as will be described below with more details. The output measurement data/signals 110p of the light detector 110, is indicative of the detected light intensity reaching the surface 110f of the light detector 110, and is coupled either by wires, or wirelessly, to a processing unit 20. Optionally, but in some embodiments preferably, the light source 100 is also connected to the processing unit 20, in order to exchange calibration signals 110c therebetween e.g., to calibrate the optical power of the emitted light 100t, or for other purposes.

The processing unit 20 comprises in some embodiments a blood volume pulse wave detection module 10v configured and operable to receive and process the measurement data/signals 110p generated by the light detector 110, extract therefrom blood volume pulse wave signals, and generate blood volume pulse wave data indicative thereof. The processing unit 20 also comprises in some embodiments a blood flow pulse wave detection module 10f configured and operable to process the measurement data/signals 110p to extract/determine therefrom a blood-flow measure indicative of the blood flow pulse wave signals, and/or of the changes in the blood flow, through the examined tissue/body part 200, and generate blood flow pulse wave data indicative thereof.

Optionally, but in some embodiment preferably, the blood flow pulse wave detection module 10f is configured to determine the blood flow pulse wave data/measure based on changes in the spectral width of the power spectrum of the detected light intensity data/signals 110p generated by the light detector 110, or on the peak of the power-spectrum of the detected light intensity data/signals 110p (c.g. in the vicinity of the light modulation frequency f0), or on the phase of the detected light intensity data/signals 110p relative to the phase of the illuminating light 110t produced by the light source 100, or on the speckle contrast, or on the decay time constant of the autocorrelation of the light intensity data/signal 110p in embodiments utilizing diffuse correlation spectroscopy (DCS), or on other parameters related to the blood flow as known in the art.

When using optical assemblies in order to measure blood flow velocities in the range of 0.1-1 m/sec, the sample rate of the optical signal should exceed 100 Hz. When the above blood flow measures are extracted at a rate higher than the cardiac pulsation rate (i.e., higher than 4 Hz), the pulsating blood flow velocity (PBFv) can be calculated according to possible embodiments hercof. For example, the PBFv can be calculated by the blood flow pulse wave detection module 10f by monitoring the peak of the power spectrum of the light, or by the full width half max (FWHM) of the power spectrum of the optical signal e.g., when using laser Doppler flowmetry. For example, a short time Fourier transform can be calculated every 1-200 ms by the spectral analysis module 10t, and the value of the transform peak can be recorded by the blood flow pulse wave detection module 10f as a function of time for thereby determining the blood flow pulse wave data.

The blood pressure (BP), which can be defined as either systolic, or diastolic, or mean arterial pressure, or as changes in the blood pressure, can be extracted by the BP module 10q from the (continuous) optical intensity data/signal 110p detected by the light detector 110, or by using an auxiliary sensor device 300. The auxiliary sensor device 300 can be implemented by any suitable type of conventional invasive or a non-invasive sensor of blood pressure, and/or of changes in the blood pressure, that is known in the art.

Optionally, but in some embodiments preferably, the blood pressure, and/or changes in the blood pressure, is determined by the BP module 10q from the measurement data/signal 110p from the light detector 110 e.g., based on the blood volume pulse wave data (PPG) and/or on the blood flow pulse wave data (e.g., pulsating blood flow velocity-PBFv), as described hereinabove or hereinbelow (e.g., as disclosed by M. Ghijsen et al (referenced hereinabove), or based on both or at least one of the blood volume (e.g., PPG) and flow (e.g., PBFv) pulsc wave data.

BP-measures indicative of the blood pressure can be extracted by the BP module 10q for example from the slope of the blood volume pulse wave (e.g., PPG) data (or of the blood flow pulse wave (e.g., PBFv) data e.g., near their main and/or secondary peaks, the second derivative of the blood volume pulse wave (e.g., PPG) data (or of the blood flow pulse wave e.g., PBFv data),or based on Acceleration Photoplethysmogram data, or directly from the measurement data/signal 110p, or from other measures.

Additionally or alternatively, the BP-measure indicative of the blood pressure of the examined subject can be extracted by the BP module 10q from the pulse arrival time (PAT) between the R peak of the QRS complex obtained from electrocardiogram (ECG) and the peak of the PPG (or of the PBFv), or alternatively from the pulse transition time (PTT) obtained from two different PPG (or two PBFv) signals recorded in different body locations of the examined subject, using conventional techniques know in the art.

Accordingly, the auxiliary sensor device 300 may comprise an ECG sensor, and/or another PPG (or PBFv) sensor. The BP module 10q of the processing unit 20 can be accordingly configured to process and analyze the data/signals from both the ECG and PPG (or PBFv) sensors of the auxiliary sensor device 300, in order to determine the time delay between the signals acquired by the two sensors, and calculate based thereon a respective BP-measure. Optionally, the auxiliary sensor 300 and the display and communication unit 30 are configured to communicate measurement and/or control data/signals over the communication channel 30e e.g., over a serial or parallel communication bus, or wirelessly.

It is noted that any of the techniques known in the art for extracting physiological measures/parameters from blood volume pulse waves (e.g., PPG) data/signals can be mutatis mutandis used to similarly extract the same measures/parameters from the blood flow pulse wave (e.g., PBFv) data/signals. Accordingly, in possible embodiments the optical measurement setup comprising the light source 100 and the light detector 110 is configured to implement a PPG sensor.

According to a possible embodiment, blood pressure is calculated by the BP module 10q in a single body location of the examined subject by extracting different features directly from the measured optical data/signals 110p, or from the blood volume pulse wave (e.g., PPG) data/signal extracted therefrom. FIG. 6 demonstrates an example of a blood volume related pulse wave (e.g., PPG) signal 1000 (normalized amplitude) as a function of time. The blood volume related pulse wave/PPG signal 1000 seen in FIG. 6 contains two (2) pronounced/distinct peaks, the main peak 1050 and the secondary peak 1060. The timing of the two (2) peaks can be calculated/identified by the BP module 10q using any suitable peak detection algorithm known in the art, and the time interval ΔPTT between the main and secondary peaks, 1050 and 1060, can be calculated by the BP module 10q as the difference between the time stamp of the secondary peak 1060 and of the main peak 1050. The relationship between the time interval/difference ΔPTT and the systolic and diastolic blood pressures depends on several parameters and models.

If the physiological parameters and/or models are known, or can be calibrated during the calibration state of the PPG sensor (e.g., implemented by the light source 100 and the light detector 110, and/or in the auxiliary sensor 300), succeeding measurements of ΔPTT, following calibration, can be used to extract the systolic and/or the diastolic blood pressures by the processing unit 20 in a continuous/periodic or semi-continuous manner. The heart rate of the examined subject can be accordingly extracted by the processing unit 20 from the time differences between consecutive main peaks 1050 (and/or secondary peaks 1060), and/or from spectral analysis of the measured optical data/signals or from the PPG signal, carried out by the spectral analysis module 10t.

Measures of either of the following: blood pressure, blood flow, PBFv, PPG, blood volume, heart rate, pulse pressure, respiratory rate, and/or any other signals that are measured by the measurement system 17 can be determined by the processing unit 20, and if needed communicated to the display and communication unit 30 e.g., using electrically conducting wires or wirelessly. The correlation module 10n of the processing unit 20 can be also configured to calculate a correlation measure corresponding to the correlation (Corr) between the determined blood flow or blood volume signals or pulse wave signals and the determined blood pressure measure. This correlation-measure corresponds to vascular reactivity or autoregulation, depending on the location of measurement on the examined subject. The correlation can be calculated by the correlation module 10n over a predetermined time period and displayed or communicated by the display and communication unit 30.

The display and communication unit 30 can be implemented by a dedicated display device configured for graphical display (e.g., of graphs, plots), a numerical display configured for display of numbers and/or symbols/characters (e.g., a liquid crystal display-LCD), or a device configured for only audible/sound outputs. The display and communication unit 30 can also utilize a general display device (e.g., touchscreen) of an electronic device (e.g., a smart device, such as smartphone, tablet, laptop, or suchlike) that can display images, graphs or numbers and sounds. Alternatively, the display and communication unit 30 does not include any display device, and only communicates the determined measurement data/signals either over wires or wirelessly to a central display, processing, storage or control system 111. The measurement data/signals information can be further processed and communicated by such central display/processing/storage/control system 111 back to the communication and display unit 30 for further evaluation by the user.

A measure of the correlation between the blood flow signals and the blood pressure measure signals, or between changes in the blood flow and blood pressure, can be determined by the module 10n of the processing unit 20. The correlation determined by the module 10n 17 can serve to alert the user, and/or a caregiver and/or medical personnel treating the user about a change in the perfusion state of body part 200 of the examined subject. Such a measure can alert to the onset of severe sepsis, shock, trauma, or inadequate perfusion or calculate vascular reactivity in body part 200 of the examined subject. Different alert-thresholds for each condition can be determined based on databases and models 111b e.g., storing history of past optical measurement data/signals of tissue perfusion in such states.

In addition, the module 10n can be configured to determine a measure of features of any parameters measured by the system 17, e.g., PPG, PBFv, BP or a combination of any of these parameters. Such measure can be determined by either comparing the measured/determined PPG, PBFv, and/or blood pressure calibration, parameters, or their derivatives, or a combination thereof, to a database of measurements 111b, or by data-driven algorithms that take into account multiple parameters, such as a machine-learning (ML) 111m or deep-learning (DL) 111d based algorithm, or any other method 111g for identifying a a measure of blood pressure or blood pressure parameters of the subject.

Alternatively, the module 10n can be configured to determine a measure of changes in blood flow, blood pressure, or in the correlation between these parameters or in any other parameter measured by the system 17, or a combination of any of these parameters, which can serve as an indication of changes in the clinical or physiological state of the patient. Such measure can be determined by either comparing the measured/determined blood flow, PBFv, and/or blood pressure, parameters, or their correlation, or a combination thereof, to a database of measurements 111b, or by data-driven algorithms that take into account multiple parameters, such as a machine-learning (ML) 111m or deep-learning (DL) 111d based algorithm, or any other method 111g for identifying a change in the clinical or physiological state of a patient/examined subject.

Accordingly, in possible embodiments, the central display/processing/storage/control system 111 comprises one or more processors 111p and memories 111m configured and operable to implement the ML 111m, DL 111d, and/or comparison/correlation based algorithms 111g, on the measurement data/signals 110p (e.g., received over the data communication channel 30s) thereby received and the database 111b of previously measured and diagnosed data/signals of other patients.

FIG. 2A schematically illustrates an implementation 17′ of the measurement setup/system 17 of FIG. 1, according to a possible embodiment, wherein the light source (L.S.) 100 and the light detector (L.D.) 110 are positioned on opposite sides of the body part 200 of the examined subject. In this non-limiting example the light detector 110 is configured to collect ballistic and/or scattered photons traveling through body part 200 of the examined subject. When the aperture 110a of the light detector 110 is positioned directly in front of the aperture 100a of the light source 100 (along the “line of sight”—LOS), and/or its aperture 110a is limited, the light detector 110 collects primarily ballistic photons which are not scattered by the cells and structure within the examined tissue 200.

In possible embodiments, the aperture 110a of the light detector 110 is sufficiently large, or alternatively, it is positioned at an angle (e.g., larger than 5 degrees) to the “line of sight” from the light source 100. In such embodiments, the light detector 110 primarily collects scattered photons that travel through the examined tissue 200 and vasculature, with a single or several scattering events. Scattered photons carry information about the movement of blood cells within the vasculature, and as such their phase and spectral characteristics (such as decorrelation time or speckle contrast) can be measured from the measurement data/signals 110p generated by the light detector 110 to determine a measure of the blood flow or of the PBFv. The light detector 110 may comprise a single light detection element, or an array of detector elements. The array of detectors (110) can be designed in a rectangular or annular arrangement, or any other geometrical arrangement that is beneficial for the collection of single or multiple scattered photons.

FIG. 2B schematically illustrates an implementation 17″ of the measurement setup/system 17 of FIG. 1, according to possible embodiments, wherein the light source 100 and the light detector 110 are arranged in a reflection geometry. In the reflection geometry 17″. the light source 100 and the light detector 110 are positioned at the same side of the body part 200 of the examined subject. In this configuration only scattered photons are detected, as described hereinabove, and a measure of blood flow, volume (e.g., PPG) and/or the PBFv can be extracted from the measurement data/signals 110p generated by the light detector 110.

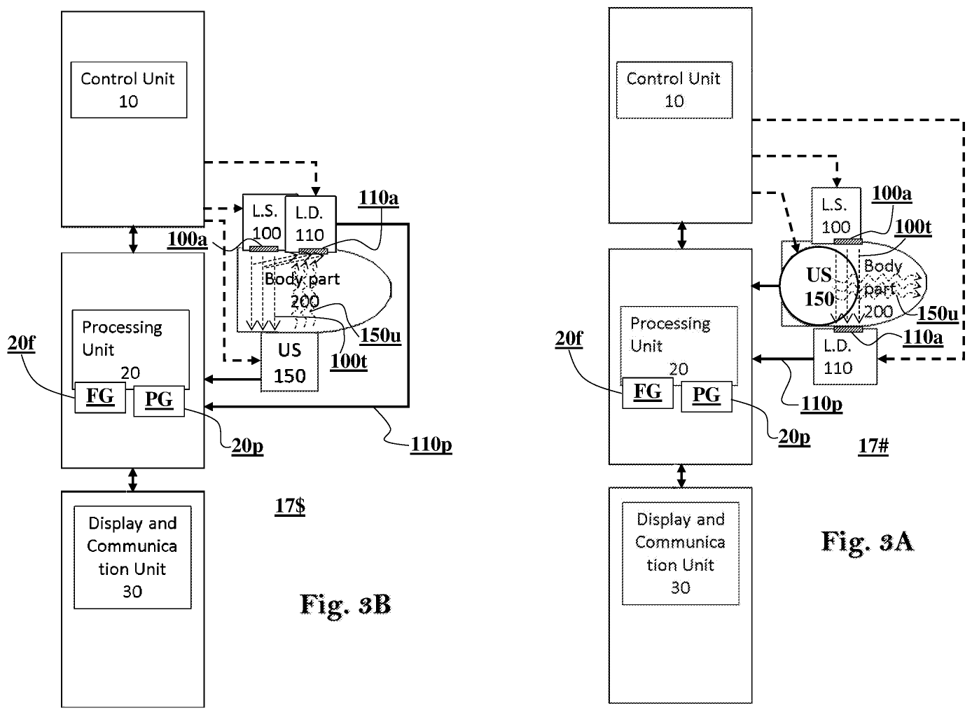

FIGS. 3A and 3B schematically illustrate systems configured for measuring blood flow parameters based on ultrasound modulation of light, wherein FIG. 3A demonstrates a system 17 #employing transmission geometry, and FIG. 3B demonstrates a system employing reflection geometry 17$. In these systems, in addition to the coherent light source 100 and the light detector 110, an ultrasound transducer 150 is coupled to the examined tissue 200. In this specific and non-limiting example, the ultrasound (US) transducer 150 is shown connected to the control unit 10. The control unit 10 comprises in some embodiments a function/signal generator (FG) 20f and/or a pulser (PG e.g., rectangular pulse signal generator) 20p configured to control the actuation of the ultrasound transducer 150.

The US-transducer 150 is configured in some embodiments to generate acoustic signals 150u at ultrasonic frequencies e.g., above 0.1 MHz, either as a continuous wave (CW), or as a coded sequence of pulses. In operation, the ultrasound waves 150u induced into the examined tissue 200 by the US-transducer 150 modulate the light 100t that propagates through the examined tissue 200, and the intensity of the light 100t reaching the light detector 110 is modulated in correlation with the generated ultrasonic signal 150u. The processing unit 20 is configured in some embodiments to process the measurement data/signals 110p generated by the light detector 110 and extract a measure of the blood flow of the examined tissue from the modulated light signal/data 110p generated by light detector 110 (for example U.S. Pat. No. 8,336,391).

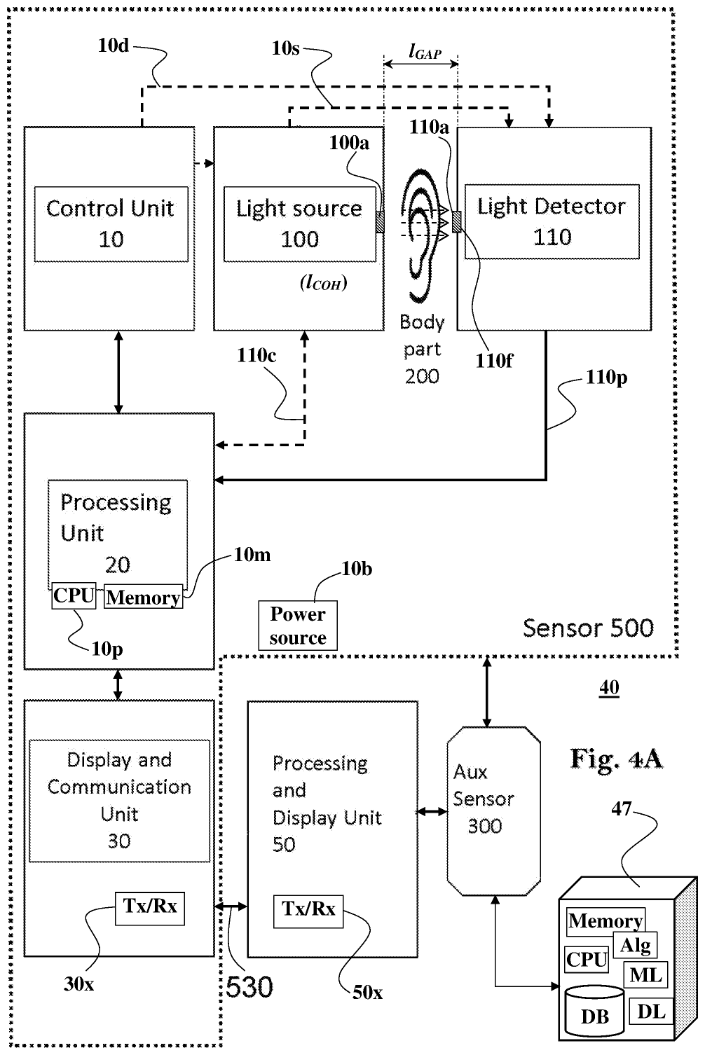

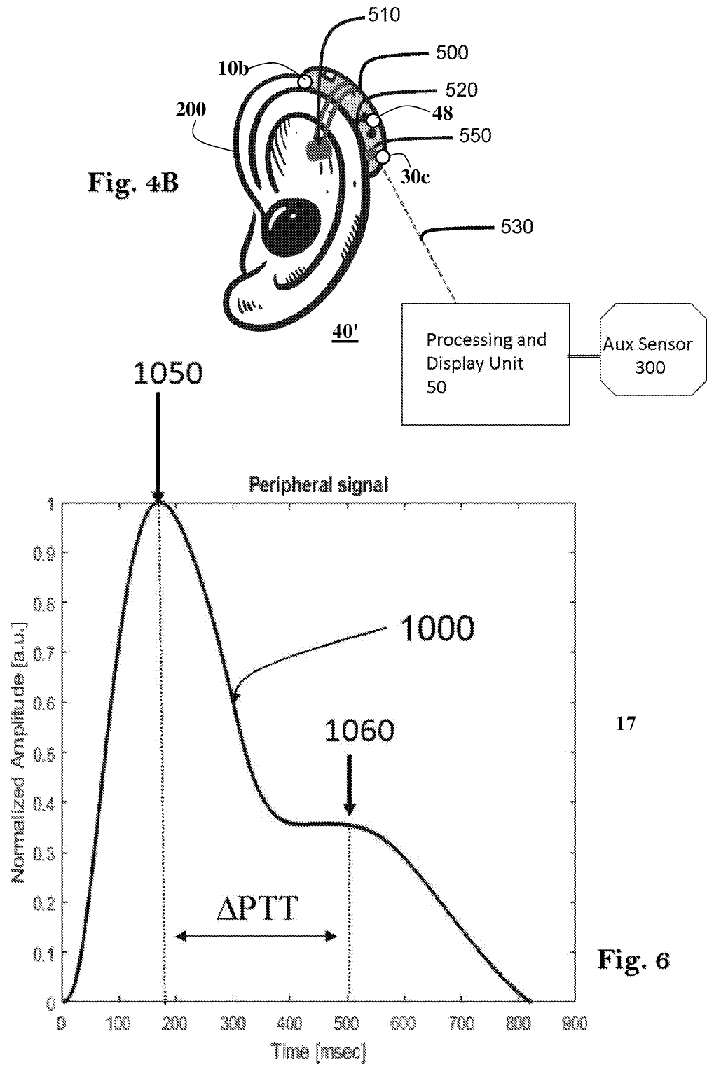

FIGS. 4A and 4B present system configurations usable for measuring blood flow parameters according to some possible embodiments. The system 40 shown in FIG. 4A is similar in many aspects to the system (17) shown in FIG. 1. but the display and processing unit 30 shown in FIG. 4A is wirelessly coupled to the sensor unit 500 (marked with dashed line). The sensor unit 500 includes the light source 100 and the light detector 110, and can also include a combination of electronic and optical elements (not shown) configured for delivering and collecting light, and further units, such as the control unit 10, and/or the processing unit 20 (and their module/units), and/or the communication unit 30, in addition to a power source (e.g., battery) 10b e.g., configured for autonomic operation of the sensor unit 500. The sensor unit 500 is configured to in some embodiments to communicate over a wireless communication channel 530 directly with the display and processing unit 50. The wireless communication channel 530 can be implemented by either a direct communication channel between the sensor unit 500 and the display and processing unit 50, or indirectly e.g., via cloud-based (e.g., over the Internet) or any other known wireless communication method or protocol.

Wireless transceivers Tx/Rx 30x,50x can be respectively implemented within the sensor unit 500 and the processing and display unit 50. The transceivers Tx/Rx 30x,50x can be configured to remotely update the software and/or firmware of the sensor unit 500 and/or the display unit 50. An auxiliary sensor 300 can be coupled, directly by wires (e.g., a serial or parallel communication bus) or wirelessly (e.g., using WiFi. Zigbee. Bluetooth, NFC, or suchlike), to the processing and display unit 50, or to the sensor unit 500, or it may be configured to independently transmit and receive data to and from a central, or cloud-based system 47. The communication and display unit 50 may also display additional information from external sensors or other such signals/data sources, in addition to those of the sensor unit 500 or of the auxiliary sensor 300.

FIG. 4B shows a specific configuration 40′ of the measurement system (40). In this example the sensor unit 500 is contained within an enclosure 520, which contains the optical and electronic clements, including an internal power source (e.g., battery) 10b and a wireless transmitter (and possibly receiver i.e., transceiver) 30c. In embodiments employing transmission geometry, the light source (100, not shown in FIG. 4B) can be operably mounted externally at 510, and the light detector (110, not shown in FIG. 4B) can be contained inside the enclosure 520, which may be the same as the enclosure of the sensor unit 500. Alternatively, the light detector (110) is externally mounted at 510, and the light source (100) is contained within the enclosure 520.

In embodiment employing reflection geometry, both the light source (100) and the light detector (110) can be externally mounted at 510, or internally mounted inside the enclosure 520. When an ultrasound transducer (150) is included in the measurement system 40′, such as exemplified in FIGS. 3A and 3B, the ultrasound transducer (150) and the light source 100 and/or the light detector 110 may be externally mounted at 510, or alternatively contained inside the enclosure 520.

A blood flow controlling clement 550 is utilized in some embodiments inside the sensor unit 500, or in an independent enclosure (not shown). The blood flow controlling element 550 can be operated by the control unit (10, not shown in FIG. 4B) to locally affect the perfusion in the tissue region being illuminated by the light source (100), and from which light reaches the detector (110). The perfusion can be locally altered by the blood flow controlling element 550 by either one of the following mechanisms: applying and releasing mechanical pressure over the tissue 200, and/or cooling or heating the tissue 200.

The heating (e.g., by an electrically resistive heating element and/or Peltier heater/cooler unit) causes vasodilation, and the cooling (e.g., by a Peltier heater/cooler unit) causes vasoconstriction. Therefore, changes to constriction and dilation properties of the vessels can be assessed based on changes in the determined PBFv, and/or based on the average blood flow velocity during or following cooling or heating. External application of pressure to the tissue 200 region, causes a drop in perfusion, and its release is commonly associated with a hyperemic response in PBFv or average blood flow velocity. The amplitude or slope of flow reduction or hyperemia serve as a measure of vascular or endothelial health. External pressure can be applied using an inflatable element e.g., cuff, sleeve, or tube (not shown) which may surround either the light source 100 or the light detector 110, or both.

In another possible embodiment, an infrared temperature sensor 48 is part of the sensor 500, or serves as an auxiliary sensor coupled 530 to sensor 500, either directly by wires or wirelessly, to the processing and display unit 50. The temperature sensor 48 can be positioned inside the car canal, and configured to continuously, periodically or repeatedly, measure body temperature from the tympanic membrane, using methods known in the art. For example, the temperature sensor 48 employs in some embodiments infra-red signals emitted from the tympanic membrane, or reflected by the tympanic membrane, or uses a thermistor in close contact with the tympanic membrane to measure its temperature. The temperature sensor 48 can be configured to transmit measurement data/signals indicative of the body temperature of the examined subject to the processing and display unit 50.

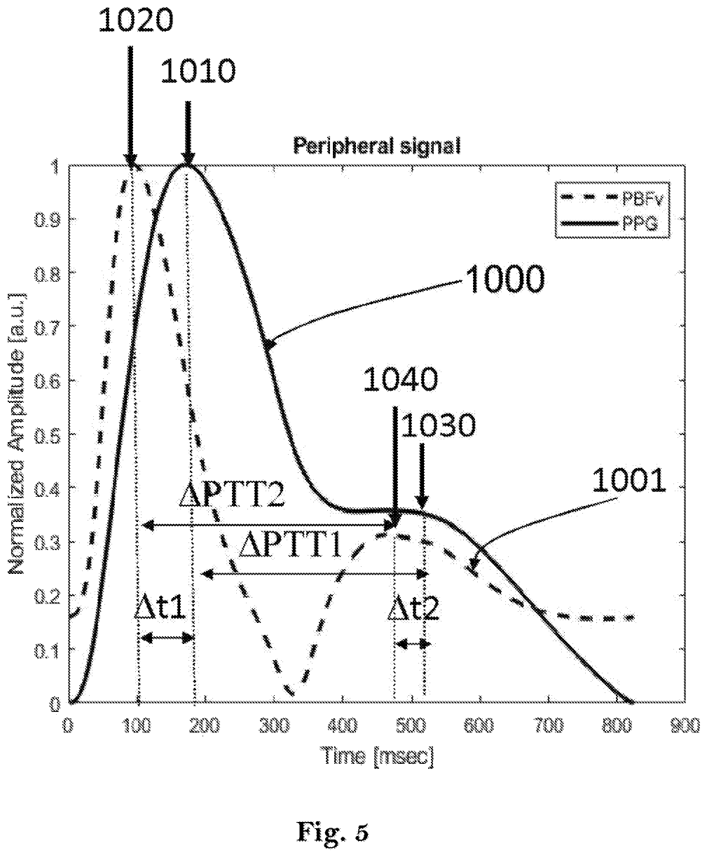

FIG. 5 shows graph plots of the blood volume related pulse wave (e.g., PPG) signal 1000 and of the blood flow related pulse wave (e.g., PBFv) signal 1001 as a function of time, where the amplitude of each signal is normalized to its main peak amplitude value, 1010 and 1020 respectively. In general, the blood volume related pulse wave (e.g., PPG) signal 1000 and blood flow related pulse wave (e.g., PBFv) signal 1001 are not normalized and correspond to the amplitude of the signals as a function of time. As seen, in the periphery, the main peak of the blood volume pulse wave/PPG signal 1000 (i.e., main peak 1010) is delayed relative to the main peak of the blood flow pulse wave/PBFv signal 1001 (i.e., main peak 1020). The time difference is also apparent in the secondary peak (peak 1030 for PPG and peak 1040 for PBFv), which is the result of reflection waves. The time difference At1 between the main peak 1010 and the main peak 1020, and/or the time difference At2 between the secondary peaks 1030 and 1030, depends on, and indicative of, vascular and hemodynamic parameters, such as compliance, resistance, blood pressure (systolic, mean and diastolic).

The un-normalized blood volume related pulse wave (e.g., PPG 1000) data/signal represents either the amplitude of a single blood volume pulse or an average of the amplitudes several (two or more) pulses. The amplitude of the PPG signal over time corresponds to the processed amplitude of the measured optical data/signal (110p), such that the amplitude of the un-normalized blood volume related pulse wave (e.g., PPG signal 1000) corresponds to the blood volume V(t) within the illuminated body part 200 using methods know in the art for calculating PPG signals (see references above). The un-normalized blood volume related pulse wave (e.g., PPG signal 1000) corresponds to changes (over time) in the volume of blood (V(t)) 5100 traveling through the examined tissue/body part (200), as equation [1] formulates:

$$V ( t ) = K 1 * PPG ( t ) \qquad [ 1 ]$$

where K1 is a constant that depends on the measurement system and can be calibrated using methods know in the art. Physically, the changes in blood volume (V(t)) 5100 over time depends on the product of the blood flow velocity (U(t)) 5200 by the cross-section area of the blood vessels (A(t)) 5300 within the sampled tissue volume in the examined body part/tissue 200. Consequently, the time derivative of the blood volume V(T) 5100 is given by equation [2], as follows:

$$\frac{d ( V ( t ) )}{dt} = A ( t ) * U ( t ) = Q ( t ) \qquad [ 2 ]$$

where Q(t) 5400 is the blood volume flow rate (volumetric flow rate). Therefore, the following expression is obtained from equations [1] and [2]:

$$\frac{d ( V ( t ) )}{dt} = K 1 * \frac{d ( PPG ( t ) )}{dt} = Q ( t ) \qquad [ 3 ]$$

The time dependent derivatives can be measured by defining a small δt such that—

$$\frac{d ( PPG ( t ) )}{dt} = \frac{PPG ( t + δ t ) - PPG ( t )}{δ t} \qquad [ 3 a ]$$

where δt is time interval, which can be as small as 1/Fs seconds (wherein Fs is the sampling rate in Hz units of the measured optical signals 110p or of the PPG(t) or PBFv(t)). Wherein U(t) corresponds to velocity extracted from the blood flow related pulse wave (e.g., PBFv) signal.

The the blood flow velocity U(t) 5200 corresponds to the blood flow related pulse wave (e.g., PBFv 5500) and is given by equation [4]:

$$U ( t ) = K 2 * PBFv ( t ) , \qquad [ 4 ]$$

where K2 is a constant that depends on the geometry of the measurement setup, and the un-normalized blood flow related pulse wave (e.g., PBFv(t)) is determined simultaneously with the un-normalized blood volume related pulse wave (e.g., PPG(t)) from the same measured optical data/signal 110p using any one of the methods/embodiments described hereinabove (e.g., based on peaks of power spectrum, FWHM, speckle decorrelation time, speckle contrast, ultrasound modulated light, etc.).

During the first phase of increase of the un-normalized blood volume related pulse wave (e.g., PPG signal 1000) and of the un-normalized blood flow related pulse wave (e.g., PBFv signal 1001), there is a linear relationship between the blood pressure and the blood flow velocity. During this phase (about quarter to half of the time between the beginning of the rise to the peak of the PBFv signal) the time dependent blood vessels cross section area A(t) 5300 can be calculated by equation [5]:

$$A ( t ) = \frac{Q ( t )}{U ( t )} = \frac{d ( K 1 * PPG ( t ) )}{dt} / ( K 2 * PBFv ( t ) ) . \qquad [ 5 ]$$

Table 3 in Nabeel et al. “Local pulse wave velocity: theory, methods, advancements, and clinical applications” (2019) IEEE Reviews in Biomedical Engineering, 13, 74-112.], describes several methods to calculate the pulse-wave-velocity (PWV). One possible method to calculate PWV(t) 6000 is given by equation [6]:

$$PWV ( t ) = \qquad [ 6 ]$$

$$\frac{dQ ( t )}{dA ( t )} = \frac{K 2 * [ \frac{d ( PPG ( t + Δ t ) )}{dt} - \frac{d ( PPG ( t ) )}{dt} ]}{[ \frac{d ( PPG ( t + Δ t ) )}{dt} / PBFv ( t + Δ t ) - \frac{d ( PPG ( t ) )}{dt} / PBFv ( t ) ]} .$$

For a short time interval Δt e.g., within the range of one (1) to hundred (100) sample points i.e., Δt∈[1,100]*1/FS, for a point in time t, M(t,t+Δt) is defined by equation [7] as follows:

$$M ( t , t + Δ t ) = \frac{[ \frac{d ( PPG ( t + Δ t ) )}{dt} - \frac{d ( PPG ( t ) )}{dt} ]}{[ \frac{d ( PPG ( t + Δ t ) )}{dt} / PBFv ( t + Δ t ) - \frac{d ( PPG ( t ) )}{dt} / PBFv ( t ) ]} . \qquad [ 7 ]$$

Therefore PWV(t)=K2*M(t,t+Δt).

From PWV(t) 6000 extracted as a function of time, changes in the blood pressure of the examined subject can be extracted using methods known in the art e.g., using equation 5 in Ma Y. et al. (“Relation between blood pressure and pulse wave velocity for human arteries”. Proc Natl Acad Sci U S A. 2018 Oct. 30; 115 (44): 11144-11149),

The time dependent blood pressure BP(t) 8000 depends on M(t,t+Δt), cab be expressed by equation [8]:

$$BP ( t ) = ε {M}^{2} ( t , Δ t ) + β \qquad [ 8 ]$$

(where BP(t) is the blood pressure signal, ϵ and β are calibration based parameters), or by equation [8a]

$$BP ( t ) = λ M ( t , Δ t ) + ξ \qquad [ 8 a ]$$

(where BP(t) is the blood pressure signal, λ and ξ are calibration based parameters).

In other embodiments normalized PPG and PBFv signals are used as the data for calculating the blood pressure in a similar method to the described above.

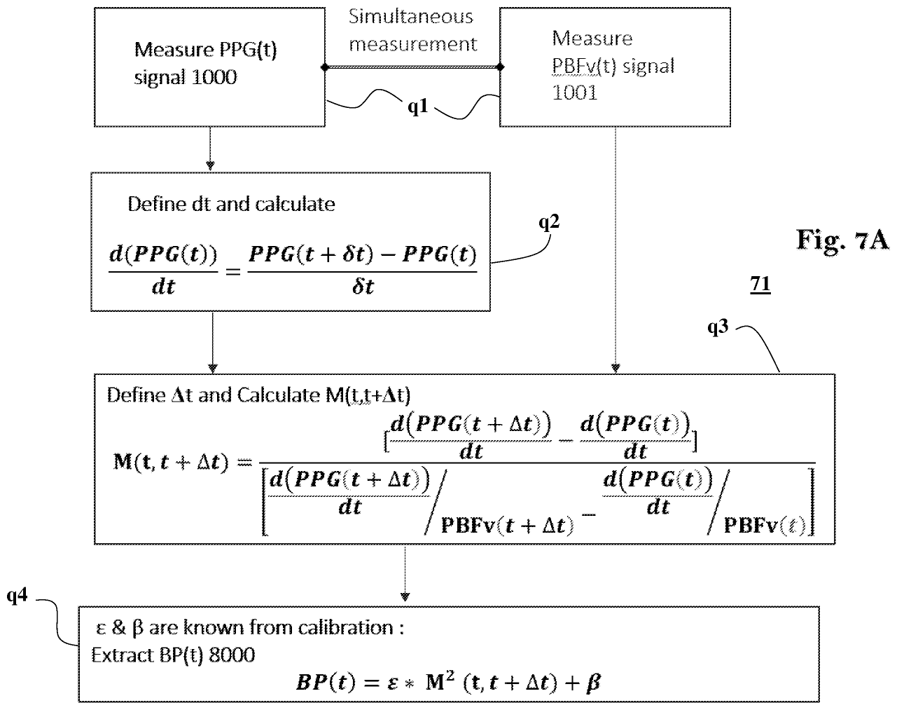

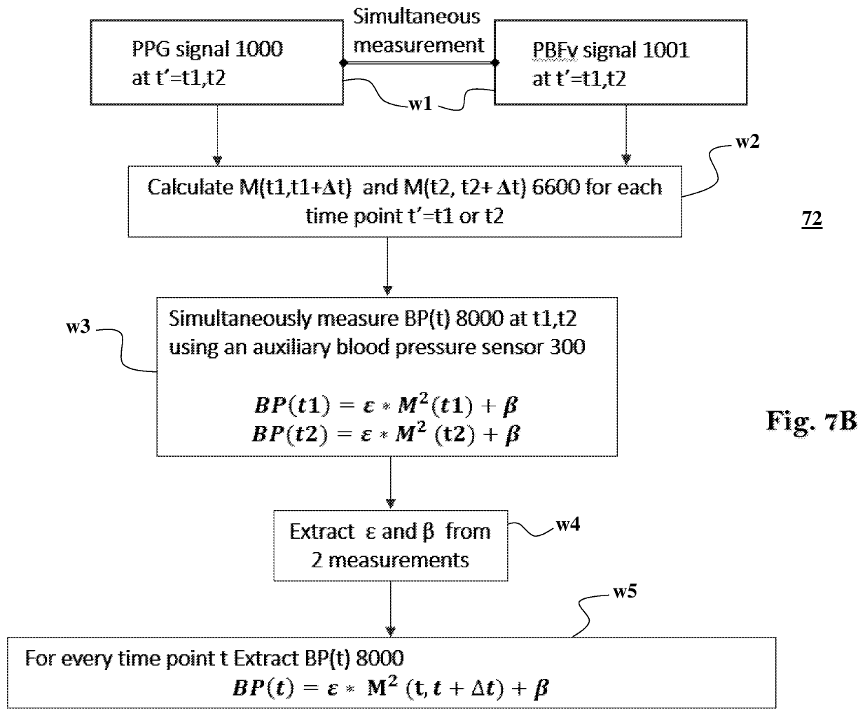

FIG. 7A is a flowchart of a process 71 for calculating blood pressure BP 8000 from two simultaneous measurements of un-normalized blood volume related pulse wave (e.g., PPG(t) 1000) signals and un-normalized blood flow related pulse wave (e.g., PBFv(t) 1001) signal. The process 71 of FIG. 7A comprises steps q1, q2, q3 and q4, which are self-explanatory, and thus will not be discussed herein in details.

It is noted that a single pulse wave, or an average of more than two such pulse waves, can be used for this measurement. For each time point t (or at each time window corresponding to PBFv(t) sample rate), or at each time point that a measurement is determined, the time derivative of the blood volume related pulse wave (e.g., PPG(t)) signal is calculated according to equation [3a]. For each timepoint t (e.g., within the time range that corresponds to the linear relation between pressure and blood flow velocity within the blood vessel defined above, but not limited to that range), M(t.t+Δt) can be calculated using equation [7], where Δt is defined hereinabove. When the calibration factors of ϵ and β are known, the blood pressure can be calculated for each time point t using equation [8] (or equation [8a]).

A possible calibration process 72 for calibrating ϵ and β is schematically illustrated in the flowchart shown in FIG. 7B. The process 72 of FIG. 7B comprises steps w1, w2, w3, w4 and w5, which are self-explanatory, and thus will not be discussed herein in details. The same calibration process 72 can be applied to calibrate λ and ξ according to equation [8a].

The blood volume related pulse wave (e.g., PPG(t)) signal and the blood flow related pulse wave (e.g., PBFv(t)) signal are first measured, either as single pulse or as an average of serval pulses. M(t.t+Δt) is then calculated for at least two different time points (t1 and t2) using equation [7]. During these same time points the blood pressure BP 8000 is measured by using an auxiliary blood pressure sensor 300 (either invasively or non-invasively). Consequently, BP(t1) is the blood pressure of the examined subject measured at time point t1, and BP(t2) is the blood pressure of the examined subject measured at time point t2. M(t1,t1+Δt) is calculated at time point t1, and M(t2,t2+Δt) is calculated at t2. From these four independent measurements, using equation [8], & and B can be extracted using linear algebra. Once they are calibrated, BP(t) 8000 can be calculated at every time point t by calculating M(t,t+Δt) from measured blood volume and flow related pulse wave signals (e.g., PPG(t) and PBFv(t)), as describe in FIG. 7A.

Other methods can be similarly used, such as described in R. Mukkamala et al., (“Toward ubiquitous blood pressure monitoring via pulse transit time: Theory and practice,”) IEEE Trans. Biomed. Eng., vol. 62, no. 8, pp. 1879-1901. June 2015), or using any other method known in the art.

Alternatively, a measure of the impedance Z 7000 from the blood flow velocity U(t) and the volumetric flow (Q(t)) can be obtained by calculating the spectral components of each signal (scc e.g., Rabben et al. (2004) “An ultrasound-based method for determining pulse wave velocity in superficial arteries”, Journal of biomechanics, 37 (10). 1615-1622).

In another embodiment the PWV is independently extracted (e.g., by the processing unit 20) utilizing two (2) sensors devices 500 positioned over adjacent locations of the examined body part/tissue (200) e.g., finger. toe, arm, calf, thigh. From the time delay t11 between the first/main peak (1010) of the blood volume pulse wave (e.g., PPG) signal in each location, and/or the corresponding delay time t10 between the first/main peak (1020) of the blood flow pulse wave (e.g., PBFv) signal, and the distance D10 between the two sensors 500. the processing unit 20 can calibrate the PWV, as PWV=D10/t10 or PWV=D10/111.

Referring back to FIG. 5. the relationship between the time differences Δt1 and Δt2 (for example their ratio, difference etc.) is used in some embodiments to calculate (e.g., by the processing unit 20) a measure of the hemodynamic or physiological properties of the vasculature. Such measures depend on the elasticity and compliance of the blood vessels, and the bifurcating vascular tree following the measurement point. The time difference ΔPTT1 is calculated in some embodiments by the processing unit 20 as the difference between the time stamp of the primary peak 1010 and of the secondary peak 1030 of the blood volume pulse wave (e.g., PPG signal) 1000, and the time difference ΔPTT2 is calculated in some embodiments (e.g., by the processing unit 20) as the difference between the time stamp of the primary peak 1020 and of the secondary peak 1040 of the PBFv signal 1001. These measures and the relationship between them are used in some embodiments to calculate (e.g., by the processing unit 20) the blood pressure, and/or other hemodynamic parameters, in addition to physiological properties of the vasculature.

Based on models of the vascular system (see, e.g., Charlton, P. H. et al. (2019), “Modeling arterial pulse waves in healthy aging: a database for in silico evaluation of hemodynamics and pulse wave indexes”. American Journal of Physiology-Heart and Circulatory Physiology, 317 (5). H1062-H1085.) the time difference At1 is demonstrated to depend on blood pressure. Therefore, following initial calibration e.g., using a gold standard blood pressure measurement system, the blood pressure can be measured based on the calculated time difference At1. Based on vascular models, or based on a database (DB in FIGS. 1 and 4A) or a machine learning system (ML and/or DL in FIGS. 1 and 4A), the relationship between systolic diastolic and mean blood pressure can be applied to extract each value.

Other features of the PPG signal 1000 and the PBFv signal 1001, such as their slopes, derivatives, correlation or cross correlation, are used in some embodiments to extract a measure of blood pressure, and/or other hemodynamic or physiological properties of the vascular system, including for example, stressed and unstressed volumes, cardiac output, stroke volume, compliance, dilation or constriction, elasticity, wall stress, wall thickness, endothelial function and properties etc.

According to one embodiment, the light source 100 comprises at least one coherent light source, that emits light at at least two different wavelengths, or at least two coherent light sources, each of which configured to emit light at a single wavelength. Each light source can be operated during a different time period/interval (e.g., shorter than 100 msec), and the light that passes though the examined tissue 200 is detected by at least one detector, and the blood volume related pulse wave (e.g., PPG) signal 1000, and the blood volume related pulse wave (e.g., PBFv) signal 1001 (see FIG. 5) are measured/determined as a function of time.

Alternatively, at least two different detectors, each having a bandpass filter, either optical, electronic in the hardware or programmed (not shown), corresponding to the separate light sources used, which are operated simultaneously, are used to detect the light intensity at cach wavelength. Oxygen saturation is calculated in some embodiments from the PPG signal measured at each wavelength using methods known in the art. In addition, the peak amplitude of the blood flow related pulse wave (e.g., PBFv) signal at each wavelength, the peak of the blood volume related pulse wave (e.g., PPG) signals at the corresponding wavelengths, or the first or second derivatives of the two signals at each wavelength, or any other corresponding measure is calculated in some embodiments e.g., by the processing unit (20) for each wavelength (collectively termed “features of dual wavelength”). Such features of dual wavelength are then used by a model, or by a machine-learning based algorithm, to extract a measure corresponding to hemodynamic or physiological properties of the vascular system.

In another possible embodiment, the sensor unit 500 is designed to be coupled to a digit. In such a configuration, the light source 100 and the light

It should also be understood that throughout this disclosure, where a process or method is shown or described, the steps/acts of the method may be performed in any order and/or simultaneously, and/or with other steps/acts not illustrated/described herein, unless it is clear from the context that one step depends on another being performed first. In possible embodiments not all of the illustrated/described steps/acts are required to carry out the method.

The flowcharts and block diagrams in the different depicted embodiments illustrate the architecture, functionality, and operation of some possible implementations of apparatuses and methods according to an illustrative embodiment. In this regard, each block in the flowcharts or block diagrams may represent a module, segment, function, and/or a portion of an operation or step. For example, one or more of the blocks may be implemented as program code, in hardware, or as a combination of the two. When implemented in hardware, the hardware may, for example, take the form of integrated circuits that are manufactured or configured to perform one or more operations in the flowcharts or block diagrams.

Functions of the system described hereinabove may be controlled through instructions executed by a computer-based control system which may be part of the control unit (10) and/or the processing unit (20). A control system suitable for use with embodiments described hereinabove may include, for example, one or more processors connected to a communication bus, one or more volatile memories (e.g., random access memory-RAM) or non-volatile memories (e.g., Flash memory). A secondary memory (e.g., a hard disk drive, a removable storage drive, and/or removable memory chip such as an EPROM, PROM or Flash memory) may be used for storing data, computer programs or other instructions, to be loaded into the computer system.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.), or embodiments combining software and hardware aspects. Furthermore, aspects of the present application may take the form of a computer program product embodied in one or more computer readable medium(s) having computer readable program code embodied thereon. The software which implements aspects of the invention can be stored on a media e.g., magnetic media, such as diskette, tape or fixed disk, or optical media, such as a CD-ROM, DVD. Additionally, the software can be supplied via the Internet or some type of private data network.

To illustrate the interchangeability of hardware and software, items such as the various illustrative blocks, modules, elements, components, methods, operations, steps, and algorithms have been described generally in terms of their functionality. Whether such functionality is implemented as hardware or software depends upon the particular application and design constraints imposed on the overall system. Skilled artisans may implement the described functionality in varying ways for each particular application.

In an embodiment where the invention is implemented using software, the software can be stored in a computer program product and loaded into the computer system using the removable storage drive, the memory chips or communications interfaces. The control logic (software), when executed by a control processor, causes the control processor to perform certain functions of the invention as described herein. In possible embodiments features of the invention are implemented primarily in hardware e.g., application specific integrated circuits (ASICs) or field-programmable gated arrays (FPGAs). Implementation of the hardware state machine so as to perform the functions described herein will be apparent to persons skilled in the relevant art(s). In yet another embodiment, features of the invention can be implemented using a combination of both hardware and software.

As described hereinabove and shown in the associated figures, the present invention provides setups and devices for physiological parameters monitoring and related methods. While particular embodiments of the invention have been described, it will be understood, however, that the invention is not limited thereto, since modifications may be made by those skilled in the art, particularly in light of the foregoing teachings. As will be appreciated by the skilled person, the invention can be carried out in a great variety of ways, employing more than one technique from those described above, all without exceeding the scope of the claims.

1. A physiological parameters monitoring device comprising: at least one coherent light source configured to emit light in one or more wavelengths onto a tissue of an examined subject at a measurement point; at least one light detector configured to measure intensity of light received from said tissue, and generate measurement data/signals indicative thereof; and a control unit configured to control the operation and process the measurement data/signals generated by said at least one light detector and simultaneously determine therefrom a pulsating blood flow signal and a pulsating blood volume signal, and determine from said pulsating blood flow and volume signals a blood pressure measure of the examined subject at the measurement point as a function of time.

2. (canceled)

3. The device of claim 1 wherein the control unit is configured to acquire the measurement data/signals at a sample rate greater than 100 Hz.

4. The device of claim 1 wherein the blood pressure measure is determined from the time derivative of the pulsating blood volume signal and from an amplitude of the pulsating blood flow signal measured at two separate time points

5. The device of claim 4 wherein the two separate time points are separated by at least two sample points.

6. The device of claim 1 wherein the control unit is configured to determine a pulse-wave-velocity (PWV) time function measure M(t,t+Δt), based on the determined pulsating blood flow and volume signals, and use said PWV time function measure to compute the blood pressure measure of the examined subject.

7. The device of claim 6 wherein the control unit is configured to receive blood pressure measurement data/signals generated by an auxiliary sensor device associated with a different tissue of the examined subject, and use said blood pressure measurement data/signals to determine a calibration biasing value B and a calibration factoring value & for the computation of the blood pressure measure of the examined subject from the determined PWV time function measure.

8. The device of claim 7 wherein the control unit is configured to determine the blood pressure measure of the examined subject by computation of the expression ϵ*M(t,t+Δt)²+β.

9. The device according to claim 1 comprising an ultrasound transducer configured to induce acoustic signals in the examined tissue for modulating the light received by the at least one light detector.

10. The device according to claim 1 wherein the control unit is configured to determine correlation between the determined pulsating blood flow or volume signal and the determined blood pressure measure, and determine based thereon a measure of vascular reactivity or autoregulation of the examined subject.

11. The device according to claim 10 wherein the control unit is configured to issue alerts indicative of changes in perfusion state of a body part of the examined subject based on the determine correlation.

12. The device according to claim 11 wherein the control unit is configured to issue the alerts for indicating of at least one of the following states: onset of severe sepsis, shock, trauma, and/or inadequate perfusion.

13. The device according to claim 1 wherein the control unit is configured to determine a vascular reactivity measure based on the determined blood pressure measure and the determined blood pulsating blood flow signal.

14. The device according to claim 1 wherein the control unit is configured to issue alerts indicative of changes in a clinical or physiological state of the examined subject based on one or more of the pulsating blood volume and/or flow signals, and/or the determined blood pressure measure, and/or a correlation thereof.

15. The device according to claim 14 wherein the control unit is configured to issue the alerts based on comparison of the determined pulsating or non-pulsating blood flow and/or volume signals and/or the determined blood pressure measure, or their correlation, or a combination thereof, to corresponding measurement data/signals retrieved from a database of such measurements.

16. The device according to claim 15 wherein the control unit is configured to issue the alerts based on machine-learning and/or deep-learning process utilizing the measurement data/signals recorded in the database of measurements.

17. The device according to claim 1 comprising a blood flow controlling element configured to locally affect perfusion in the examined tissue responsive to instructions from the control unit, and wherein the control unit is configured to operate said blood flow controlling element based on the determined pulsating blood flow and/or volume signals and/or the determined blood pressure measure.

18. The device according to claim 17 wherein the blood flow controlling element is configured to controllably apply pressure to the examined tissue, and/or cool or heat the examined tissue.

19. (canceled)

20. The device according to claim 1 wherein the coherent light source is configured to selectively emit the light in two or more different wavelengths, and wherein the control unit is configured to determine oxygen saturation of the examined subject based on the blood pulse wave determined for said two or more different wavelengths.

21. The device according to claim 1 configured as a wearable device.

22. A method of measuring physiological parameters of an examined subject, the comprising illuminating a tissue by at least one coherent light source; measure intensity of light received from said tissue and generating measurement data/signals indicative thereof, processing said measurement data/signals and simultaneously determining therefrom a pulsating blood flow signal and a pulsating blood volume signal, and determining from said pulsating blood flow and volume signals a blood pressure measure of the examined subject at the measurement point as a function of time.

23-37. (canceled)
